(19) United States
(12) Patent Application Publication
(10) Pub. No.: US 2012/0269770 A1
(43) Pub. Date: Oct. 25, 2012

(54) Stable Preserved Compositions of Interferon-Beta

(76) Inventors: Mark Brader, San Diego, CA (US); Anisa Vaidya, Newbury Park, CA (US)

(21) Appl. No.: 13/301,354

(22) Filed: Nov. 21, 2011

Related U.S. Application Data
(60) Provisional application No. 61/416,291, filed on Nov. 22, 2010.

(51) Int. Cl. A61K 38/21

(52) U.S. Cl. 424/85.6

(57) ABSTRACT

Stable preserved compositions of interferon-β and pegylated-interferon-β are described.

### CLAIM OF PRIORITY

This application claims the benefit of prior U.S. Provisional Application No. 61/416,291 filed on Nov. 22, 2010, which is incorporated by reference in its entirety.

### TECHNICAL FIELD

This description relates to stable preserved compositions of interferon-β and pegylated-interferon-β.

### BACKGROUND

Multiple sclerosis (MS) is one of the most common diseases of the central nervous system and affects over 2.5 million people worldwide. Treatment of multiple sclerosis include the administration of interferon-beta to a patient. For example, interferon-beta-1a is marketed in the United States under the trade names of AVONEX™ (Biogen Idec MA Inc.) and REBIF™ (EMD Serono) and interferon-beta-1b is marketed in the United States as BETASERON™ (Berlex) and EXTAVIA™ (Novartis).

Single use drug formulations of interferon do not contain a preservative. Additionally, if the sterility of the single use drug product is compromised, the drug must be used within 24 hours or else there may be significant patient safety issues with contamination from microbial growth. Current multi-use drug formulations contain benzyl alcohol which can act as a preservative and allows a series of doses to be administered from a single container. When used over long periods of time, proteins are generally physically or chemically unstable in the presence of pharmaceutical preservatives. For interferon-beta, the primary problems are degradation, aggregation and deamidation.

### SUMMARY

The composition described herein has been found to confer good stability to interferon-beta, for example, interferon-beta-1a, interferon-beta-1b, pegylated interferon-beta-1a or pegylated interferon-beta-1b.

In one embodiment, an aqueous pharmaceutical composition that includes interferon beta at a concentration of about 0.05 mg/ml to about 0.2 mg/ml, phenol at about 1 mg/ml to 6 mg/ml and sucrose at about 250 mM to 350 mM, a salt and a surfactant is described. The interferon beta can be interferon beta-1a or pegylated interferon beta-1a or interferon beta-1b. The interferon beta can be at a concentration of 0.1 mg/ml or at a concentration of 0.2 mg/ml. The interferon beta can be at a concentration of about 0.1 to about 0.2 mg/ml. The phenol can be at a concentration of about 2.5 mg/ml to about 3.5 mg/ml. The sucrose can be at a concentration of 300 mM or at a concentration of about 250 mM to 350 mM. The salt can be sodium acetate. The surfactant can be polysorbate. The pH of the composition can be about 4.5 to about 6.0. The pH of the composition can be about 4.8. The composition can be stable after freeze-thaw cycling or after agitation stress. The composition can be stable for at least 1 month at room temperature as measured by % monomer loss of interferon-beta. The composition can be stable for at least 1 month at room temperature as measured by % soluble aggregates of interferon-beta. In other embodiments, the composition can be stable for at least 1 day, 2 days, 3 days, 4 days, 5 days, 6 days, 1 week, 2 weeks, 3 weeks, 1 month or more when stored at about 2 to 25° C., at 5° C., at 10° C., at 15° C., at 20° C. or at 25° C.

The composition can be formulated as a multi-use pharmaceutical formulation.

Other features will become apparent from the following detailed description, taken in conjunction with the accompanying drawings, which illustrate by way of example, the features of the various embodiments.

### BRIEF DESCRIPTION OF THE DRAWINGS

FIG. 1 is graphic illustration of the effect of freeze-thaw stress on interferon-beta-1a formulations as measured by % monomer loss after 1 month at 5° C.

FIG. 2 is a graphic illustration of the effect of 72 hours of agitation on interferon-beta-1a formulations as measured by % monomer loss after 1 month at 5° C.

FIG. 3 a graphic illustration of the effect of room temperature on interferon-beta-1a formulations as measured by % monomer loss after 1 month at 25° C.

FIG. 4 is a graphic illustration of the effect of freeze-thaw stress on interferon-beta-1a formulations as measured by % soluble aggregates after freeze-thaw cycles after 1 month at 5° C.

FIG. 5 is a graphic illustration of the effect of 72 hours of agitation on interferon-beta-1a formulations as measured by % soluble aggregates after 1 month at 5° C.

FIG. 6 is a graphic illustration of the effect of room temperature on interferon-beta-1a formulations as measured by % soluble aggregates after 1 month at 25° C.

### DETAILED DESCRIPTION

Stable preserved compositions of interferon-beta that contain the combination of phenol and sucrose that can be used in a single dose or multi-dose formulation are described.

The term “interferon” or “IFN” as used herein means the family of highly homologous species-specific proteins that inhibit viral replication and cellular proliferation and modulate immune response. Human interferons are grouped into two classes; Type I, including alpha and beta-interferon, and Type II, which is represented by gamma-interferon only. Recombinant forms of each group have been developed and are commercially available. Subtypes in each group are based on antigenic/structural characteristics. An example of interferon used herein is a glycosylated, human, interferon-beta that is glycosylated at residue 80 (Asn 80) and that is derived via recombinant DNA technologies.

This glycosylated interferon-beta is also known as “interferon-beta-1a” or “IFN-beta-1a” or “interferon beta 1a,” all used interchangeably. The term “interferon-beta-1a” is also meant to encompass mutants thereof, provided that such mutants are also glycosylated at residue 80 (Asn 80). Recombinant DNA methods for producing proteins, including interferons are known. See for example, U.S. Pat. Nos. 4,399,216, 5,149,636, and 5,179,017 (Axel et al) and U.S. Pat. No. 4,470,461 (Kaufman).

Mutants of interferon-beta-1a may be used. Mutations are developed using conventional methods of directed mutagenesis, known to those of ordinary skill in the art and can include functionally equivalent interferon-beta-1a polynucleotides that encode for functionally equivalent interferon-beta-1a polypeptides.

The construction of recombinant DNA plasmids containing sequences encoding at least part of human fibroblast interferon and the expression of a polypeptide having immunological or biological activity of human fibroblast interferon is also contemplated. The construction of hybrid beta-interferon genes containing combinations of different subtype sequences can be accomplished by techniques known to those of skill in the art.

Typical suitable recombinant interferon-betas which may be used in the compositions described herein include but are not limited to interferon beta-1a such as AVONEX™ (Biogen Idec MA Inc.) and REBIF™ (EMD Serono) and interferon-beta-1b, marketed in the United States as BETASERON™ (Berlex) and EXTAVIA™ (Novartis).

The interferon-beta protein may be conjugated to polyalkylene glycol residues of C1-C4 alkyl polyalkylene glycols, preferably polyethylene glycol (PEG), or poly(oxy)alkylene glycol residues of such glycols. Thus, the polymer to which the protein is attached can be a homopolymer of polyethylene glycol (PEG) or is a polyoxyethylated polyol, provided in all cases that the polymer is soluble in water at room temperature. Non-limiting examples of such polymers include polyalkylene oxide homopolymers such as PEG or polypropylene glycols, or polyoxyethylenated glycols, copolymers thereof or block copolymers thereof, provided that the water solubility of the block copolymer is maintained. Examples of polyoxyethylated polyols include, for example, polyoxyethylated glycerol, polyoxyethylated sorbitol, polyoxyethylated glucose, or the like. The glycerol backbone of polyoxyethylated glycerol is the same backbone occurring naturally in, for example, animals or humans in mono-, di-, or triglycerides. Therefore, this branching would not necessarily be seen as a foreign agent in the body.

As an alternative to polyalkylene oxides, dextran, polyvinyl pyrrolidones, polyacrylamides, polyvinyl alcohols, carbohydrate-based polymers or the like may be used. Those of ordinary skill in the art will recognize that the foregoing list is merely illustrative and that all polymer materials having the qualities described herein are contemplated. The polymer need not have any particular molecular weight, but it is preferred that the molecular weight be between about 300 and 100,000, more preferably between 10,000 and 40,000. In particular, sizes of 20,000 or more are best at preventing protein loss due to filtration in the kidneys.

Polyalkylene glycol derivatization has a number of advantageous properties in the formulation of polymer-interferon-beta 1a conjugates, as associated with the following properties of polyalkylene glycol derivatives: improvement of aqueous solubility, while at the same time eliciting no antigenic or immunogenic response; high degrees of biocompatibility; absence of in vivo biodegradation of the polyalkylene glycol derivatives; and ease of excretion by living organisms. Conjugation of interferon-beta to PEG is further described in U.S. Pat. No. 7,446,173 and U.S. Application Publication No. 20050107277, both of which are herein incorporated by reference.

The compositions described herein can be formulated as aqueous pharmaceutical compositions. In the alternative, the interferon-beta described herein can be formulated in lypholized form and can include a diluent. A stable composition of interferon-beta exhibits little or no signs of any one or more of aggregation, fragmentation, deamidation, oxidation, or change in biological activity over an extended period of time, e.g., 12 months, 24 months, 36 months or longer. For example, in one embodiment, less than 10% of the composition is aggregated, fragmented, or oxidated. Aggregation, precipitation, and/or denaturation can be assessed by known methods, such as visual examination of color and/or clarity, or by UV light scattering or size exclusion chromatography. HPLC or reversed phase HPLC(RP-HPLC) or variants of HPLC may be used to measure the quantity of covalently intact and soluble protein in a composition. Loss of protein represents degradation due to instability. Other known methods that can be used are as described in Manning et al., Pharmaceutical Research, Vol. 27, No. 4, April 2010, herein incorporated by reference, may also be used to measure protein formulation stability.

The ability of the protein to retain its biological activity can be assessed by detecting and quantifying chemically altered forms of the protein. Size modification (e.g., clipping), which can be evaluated using size exclusion chromatography, SDS-PAGE and/or matrix-assisted laser desorption ionization/time-of-flight mass spectrometry (MALDI/TOF MS), or peptide mapping of endoproteinase-treated protein, for example. Other types of chemical alteration include charge alteration (e.g., occurring as a result of deamidation), which can be evaluated by ion-exchange chromatography, for example. A protein retains its biological activity in a pharmaceutical formulation, if the biological activity of the protein at a given time is within about 10% of the biological activity exhibited at the time the pharmaceutical formulation was prepared as determined in an assay.

Interferon-beta or interferon-beta-1a, for example, can be provided in a buffered solution at a concentration of about 0.1 mg/ml to about 0.9 mg/ml, of about 0.1 mg/ml to about 0.5 mg/ml, of about 0.1 mg/ml to about 0.3 mg/ml or of about 0.1 mg/ml to about 0.2 mg/ml. Interferon-beta-1a can be provided in a buffered solution at a concentration of 0.25 mg/ml. The composition can be stored at 2-25° C., at 5° C., at 10° C., at 15° C., at 20° C. or at 25° C. The composition can be stable at room temperature for 1, 2, 3, 4, or 5 days or more. Room temperature can be about 18° C., 19° C., 20° C., 21° C., 22° C., 23° C., 24° C. or 25° C.). The composition can be stored at a first low temperature, for example, less than 18° C. or from about freezing but at or below 15° C., 10° C. or 4° C. and can be stored at second higher temperature, for example, without refrigeration or at room temperature, for about 1-5 days. In other embodiments, the interferon-beta which is formulated as described below can be stable for at least 1 day, 2 days, 3 days, 4 days, 5 days, 6 days, 1 week, 2 weeks, 3 weeks, 1 month or more when stored at 2-25° C., at 5° C., at 10° C., at 15° C., at 20° C. or at 25° C.

In one embodiment, interferon-beta-1a can be formulated with phenol, sucrose, a salt, a surfactant. In another embodiment, lypholized interferon-beta-1a can be reconstituted with a diluent that includes phenol, sucrose, a salt, and/or a surfactant. The composition can be prepared without anti-oxidants. The composition can be methionine free. In one embodiment, the formulation can be prepared with phenol at a concentration between 1 mg/ml to 6 mg/ml, between 2 mg/ml to 5 mg/ml or between 2 mg/ml to 4 mg/ml. The composition can be prepared with phenol at a concentration of between 2.5 mg/ml to 3.5 mg/ml. The composition can be formulated with phenol at a concentration of about 2.5 mg/ml, 2.6 mg/ml, 2.7 mg/ml, 2.8 mg/ml, 2.9 mg/ml, 3.0 mg/ml, 3.1 mg/ml, 3.2 mg/ml. 3.3 mg/ml, 3.4 mg/ml, 3.5 mg/ml. The composition can be prepared with sucrose at a concentration of about 250 mM, 300 mM, 350 mM or 400 mM. Since sucrose may function as an isotonic agent, isotonic agent or agents may be used in amounts which impart to the solution the same or essentially the same osmotic pressure as body fluid. Other sugars such as dextrose, mannitol or lactose can be used.

The composition can include a pharmaceutically acceptable salt which refers to a salt that retains the desired biological activity of the antibody and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, or the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids, free amino acids, or the like. Base addition salts include those derived from alkaline earth metals, such as sodium, potassium, magnesium, calcium or the like, as well as from nontoxic organic amines, such as N,N′-dibenzylethylenediamine, N-methylglucamine, chloroprocaine, choline, diethanolamine, ethylenediamine, procaine or the like.

The composition can include a salt, such as sodium acetate or sodium chloride at a concentration of about 10 mM to about 200 mM. The composition can contain sodium acetate at a concentration of 20 mM, 30 mM, 40 mM, 50 mM, 100 mM, 125 mM, 150 mM or 175 mM.

In another embodiment, the composition can contain a pharmaceutically acceptable excipient, such as a surfactant, such as polysorbate 80, in an amount of about 0.001% to about 2.0%, about 0.004% to about 0.4%, about 0.008 to about 0.2%, or about 0.02% to about 0.08% (w/v) (e.g., about 0.01%, about 0.02%, about 0.03%, about 0.04%, about 0.05%, about 0.06%, about 0.07%, about 1%, or about 1.5%). Other excipients that can be used can include polyoxyethylene derivatives, Tween, Pluronic, glycerin monostearate, polyoxyl stearate, lauromacrogol, or sorbitan oleate. The pH of the composition can be about 6.0±0.5, about 5.0±0.5, about 6.0±0.5 or about 7.0±0.5. The pH of the composition can be 4.8. The pH of the composition can be about pH 4.5 to about 6.0.

Pharmaceutical compositions are sterile and stable under the conditions of manufacture and storage. A pharmaceutical composition can also be tested to insure it meets regulatory and industry standards for administration.

Exemplary conditions which can be treated with interferon include, but are not limited to, cell proliferation disorders, in particular multiple sclerosis, cancer (e.g., hairy cell leukaemia, Kaposi's sarcoma, chronic myelogenous leukaemia, multiple myeloma, basal cell carcinoma and malignant melanoma, ovarian cancer, cutaneous T cell lymphoma), or viral infections. Without limitation, treatment with interferon may be used to treat conditions which would benefit from inhibiting the replication of interferon-sensitive viruses. For example, interferon can be used alone or in combination with AZT in the treatment of human immunodeficiency virus (HIV)/AIDS or in combination with ribavirin in the treatment of HCV. Viral infections which may be treated in accordance with the invention include, but are not limited to, hepatitis A, hepatitis B, hepatitis C, other non-A/non-B hepatitis, herpes virus, Epstein-Barr virus (EBV), cytomegalovirus (CMV), herpes simplex, human herpes virus type 6 (HHV-6), papilloma, poxvirus, picornavirus, adenovirus, rhinovirus, human T lymphotropic virus-type 1 and 2 (HTLV-1/-2), human rotavirus, rabies, retroviruses including HIV, encephalitis, or respiratory viral infections.

The IFN-beta or pegylated IFN-beta is administered in a pharmacologically-effective amount to treat any of the conditions described above. The term “pharmacologically-effective amount” means the amount of a drug or pharmaceutical agent that will elicit the biological or medical response of a tissue, system, animal or human that is being sought by a researcher or clinician. It is an amount that is sufficient to significantly affect a positive clinical response while maintaining diminished levels of side effects. The amount of IFN-beta which may be administered to a subject in need thereof is in the range of 0.01-100 μg/kg, or more preferably 0.01-10 μg/kg, administered in single or divided doses. Administration of the described dosages may be every other day, but preferably occurs once a week or once every other week. Doses are administered over at least a 24 week period by injection. The dosage regimen utilizing the composition described herein is selected in accordance with a variety of factors including type, species, age, weight, sex and medical condition of the patient; the severity of the condition to be treated; the route of administration; the renal and hepatic function of the patient; or the particular compound or salt thereof employed. The activity of the interferon-beta and sensitivity of the patient to side effects are also considered. An ordinarily skilled physician or veterinarian can readily determine and prescribe the effective amount of the drug required to prevent, counter or arrest the progress of the condition.

In yet another embodiment, the composition is suitable for subcutaneous or intramuscular administration. In even another embodiment, the composition is suitable for IV administration. The compositions described herein can be administered by a parenteral mode (e.g., subcutaneous, intraperitoneal, or intramuscular injection). The phrases “parenteral administration” and “administered parenterally” as used herein mean modes of administration other than enteral and topical administration, usually by injection, and include, subcutaneous or intramuscular administration, as well as intravenous, intracapsular, intraorbital, intracardiac, intradermal, intraperitoneal, transtracheal, subcuticular, subcapsular, subarachnoid, intraspinal, epidural and intrasternal injection or infusion. Administration of the dose can be intravenous, subcutaneous, intramuscular, or any other acceptable systemic method. Based on the judgment of the attending clinician, the amount of drug administered and the treatment regimen used will, of course, be dependent on the age, sex and medical history of the patient being treated, the neutrophil count (e.g., the severity of the neutropenia), the severity of the specific disease condition and the tolerance of the patient to the treatment as evidenced by local toxicity or by systemic side-effects.

Parental injectable administration is generally used for subcutaneous, intramuscular or intravenous injections and infusions. For example, when a subcutaneous injection is used to deliver 0.01-100 μg/kg, or more preferably 0.01-10 μg/kg of PEGylated IFN-beta over one week, two injections of 0.005-50 μg/kg, or more preferably 0.005-5 μg/kg, respectively, may be administered at 0 and 72 hours. Additionally, one approach for parenteral administration employs the implantation of a slow-release or sustained-released system, which assures that a constant level of dosage is maintained, according to U.S. Pat. No. 3,710,795, incorporated herein by reference.

Lyophilized compositions for injections can, for example, be prepared by dissolving, dispersing, etc. The active compound is dissolved in or mixed with a pharmaceutically-pure solvent such as, for example, water, saline, aqueous dextrose, glycerol, ethanol, or the like, to form the injectable solution or suspension. Additionally, solid/lyophilized forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically-valuable substances.

Pharmaceutical compositions can be administered with medical devices. For example, pharmaceutical compositions can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. The therapeutic composition can also be in the form of a biodegradable or nonbiodegradable sustained release formulation for subcutaneous or intramuscular administration. See, e.g., U.S. Pat. Nos. 3,773,919 and 4,767,628 and PCT Application No. WO 94/15587. Continuous administration can also be achieved using an implantable or external pump. The administration can also be conducted intermittently, e.g., single daily injection, or continuously at a low dose, e.g., sustained release formulation. The delivery device can be modified to be optimally suited for administration of interferon-beta. For example, a syringe can be siliconized to an extent that is optimal for storage and delivery of interferon-beta. Of course, many other such implants, delivery systems, and modules are also known.

### EXAMPLES

Formulation test samples containing interferon beta-1a (100 mg/ml), sucrose (300 mM), phenol (either 2.5 mg/ml or 3.5 mg/ml), polysorbate 20 (0.005% w/v), sodium acetate (20 mM), pH 4.8 were prepared from a stock solution of 0.3 mg/mL Interferon β-1a in 20 mM acetate buffer. Similarly, comparator compositions corresponding to the REBIF™ and AVONEX™ formulation compositions were prepared, also at an interferon beta-1a concentration of 100 mg/ml. Comparator compositions corresponding to REBIF™ formulation compositions further contain 10 mM sodium acetate, 247 mM mannitol, 0.8 mM L-methionine, 0.05% w/v poloxamer 188, 46 mM benzyl alcohol at a pH of 4.0. Comparator compositions corresponding to AVONEX™ formulation compositions contain 20 mM sodium acetate, 150 mM L-arginine HCl, 0.005% w/v polysorbate 20, at a pH of 4.8.

All solutions were sterile-filtered using 0.22 μm vacuum filtration units (Corning P/N 431153, 431096, 431097 and 431098, and Millipore P/N SGP00525). These formulations were prepared in a sterile hood by transferring appropriate volumes of excipient stock solution. pH values were adjusted to target values using 0.1M NaOH or 1M HCl. Final formulated solutions were sterile-filtered using 0.22 μm Millipore 50 ml disposable vacuum filtration units (P/N SGP00525). Aliquots were transferred (2 ml for each formulation) into autoclaved 5 ml vials (20 mm wide-neck, glass type 8412-G, West Pharmaceutical Services, P/N 68000318) and autoclaved stoppers (20 mm Florotec, West P/N 19700022) in a sterile hood. After capping all vials, they were transferred from the hood and crimped, using color-coding for each formulation. The following composition was used in all the experiments described below: interferon beta-1a (100 μg/ml), sucrose (300 mM), phenol (either 2.5 mg/ml or 3.5 mg/ml), polysorbate 20 (0.005% w/v), sodium acetate (20 mM), pH 4.8.

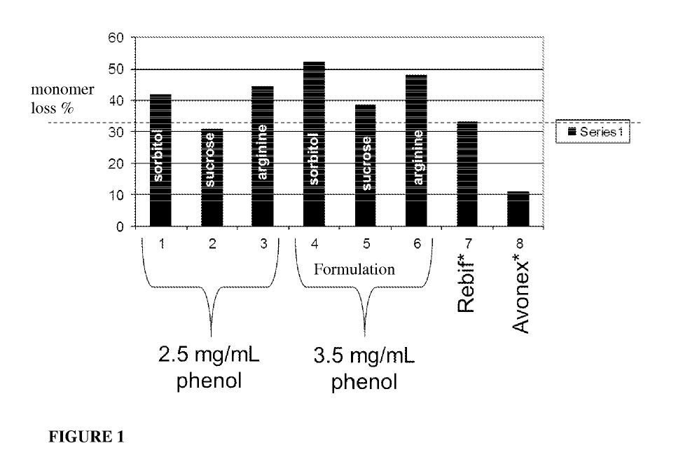

FIG. 1 shows the effect of freeze-thaw stress on the stability of trial formulations relative to the comparator formulations corresponding to commercial formulations of AVONEX™ and REBIF™ as measured by % monomer loss after freeze-thaw cycling and one month of storage at 5° C. FIG. 1 shows the destabilizing effect of preservatives. AVONEX™ is the only formulation shown in FIG. 1 with no preservative present. The data demonstrates that in the presence of phenol, sucrose is a more effective stabilizer than sorbitol or arginine.

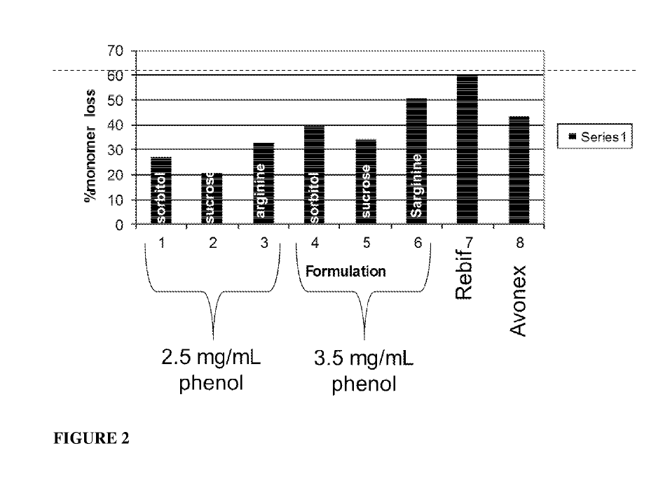

Samples of each formulation were set up on a rotational shaker at 650 rpm for a 72 hour agitation study at ambient temperature. FIG. 2 demonstrates the effect of agitation stress on the stability of trial formulations relative to the comparator formulations corresponding to commercial formulations of AVONEX™ and REBIF™ as measured by % monomer loss after 72 hours of agitation and one month of storage at 5° C. The data demonstrates that in the presence of phenol, sucrose is a more effective stabilizer than sorbitol or arginine. The data further shows that sucrose-phenol compositions were more stable than comparator formulations corresponding to AVONEX™ and REBIF™ formulations.

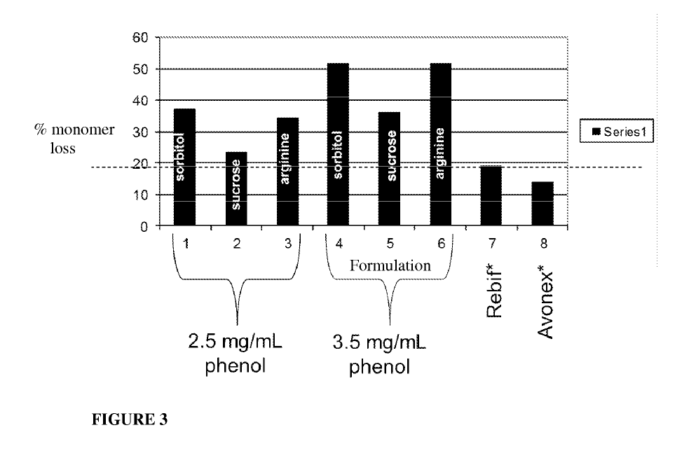

Samples of each formulation were stored at 5, 25 and 40° C. FIG. 3 demonstrates the effect of room temperature on the stability of trial formulations relative to the comparator formulations corresponding to commercial formulations of AVONEX™ and REBIF™ as measured by % monomer loss after 1 month at 25° C. The data demonstrates that in the presence of phenol, sucrose is a more effective stabilizer than sorbitol or arginine.

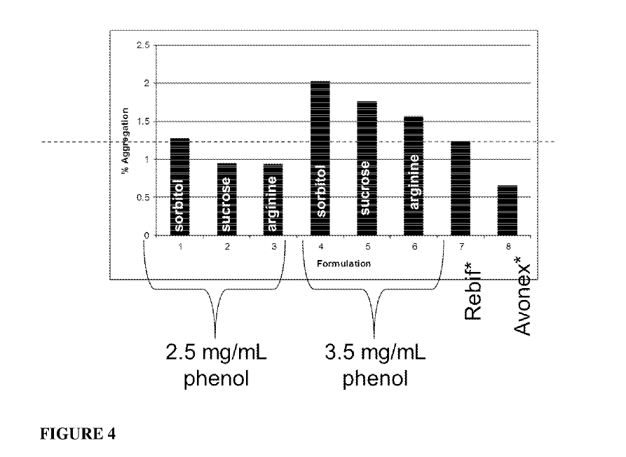

Samples were subjected to 5× freeze/thaw cycles (−70° C. for freezing, ambient temperature for thawing) over a 1 week period. Time points are taken and analyzed at T=0, 1 week, 2 weeks, 1 month and 3 months. FIG. 4 shows the effect of freeze-thaw stress on the stability of trial formulations relative to the comparator formulations corresponding to commercial formulations of AVONEX™ and REBIF™ as measured by % soluble aggregates after freeze-thaw cycling and one month of storage at 5° C. The data demonstrates that in the presence of phenol, sucrose is a more effective stabilizer than sorbitol or arginine.

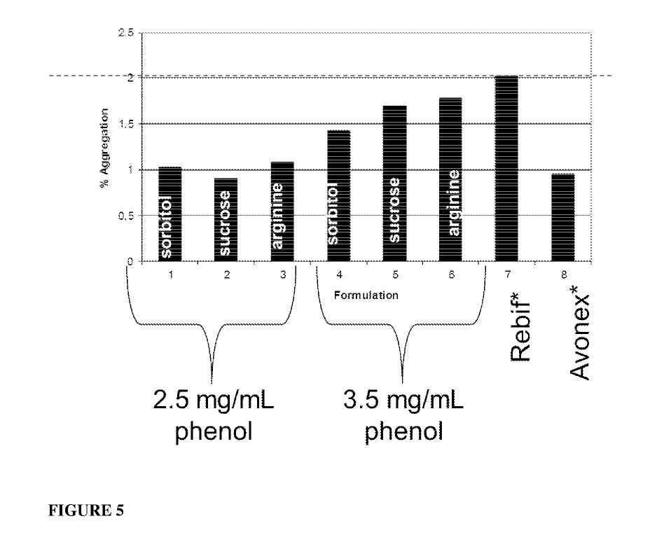

Samples of each formulation were set up on a rotational shaker at 650 rpm for a 72 hour agitation study at ambient temperature. FIG. 5 demonstrates the effect of agitation stress on the stability of trial formulations relative to the comparator formulations corresponding to commercial formulations of AVONEX™ and REBIF™ as measured by % soluble aggregates after 72 hours of agitation and one month of storage at 5° C. The data demonstrates that in the presence of phenol, sucrose is a more effective stabilizer than sorbitol or arginine.

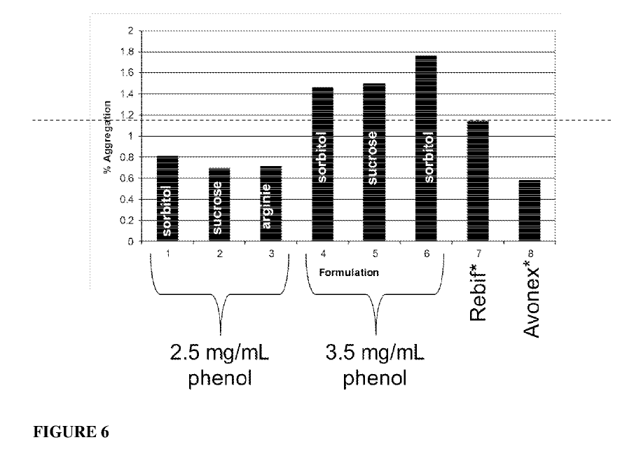

Samples of each formulation were stored at 5, 25 and 40° C. FIG. 6 demonstrates the effect of room temperature stress on the stability of trial formulations relative to the comparator formulations corresponding to commercial formulations of AVONEX™ and REBIF™ as measured by % soluble aggregates after 1 month at 25° C. The data demonstrates that in the presence of phenol, sucrose is a more effective stabilizer than sorbitol or arginine. The data also suggests that at levels towards the lower end of the 2.5 to 3.5 mg/ml phenol range, sucrose-phenol formulations may be more stable than the comparator formulation corresponding to the commercial formulation of REBIF™.

Formulations were prepared by adding appropriate quantities of excipients to an interferon-beta-1a stock solution in 20 mM actetate buffer, to achieve the compositions listed in Table 1. Table 1 demonstrates the stabilizing effect of stabilizers that include sucrose, mannitol, glycine, arginine, arginine HCl, sorbitol, sodium sulfate, polysorbate 20, sodium chloride on interferon-beta-1a in the presence of different preservatives such as phenol and m-cresol. The stability of interferon-beta-1a was measured based on protein concentration using RP-HPLC after 0 days, 4 days or 24 days at 25° C. Quantification was based on the fact that unstable formulations lose protein concentration. In this stabilizer screen, 20 mM IFN-beta-1a in sodium acetate, pH 4.8 was used. The following concentrations of stabilizers were used: 50 mg/ml sucrose, 50 mg/ml mannitol, 50 mg/ml glycine, 150 mM arginine, 150 mM arginine HCl, 25 mg/ml sorbitol, 75 mM sodium sulfate, 1 mg/ml polysorbate 20 (0.1% w/v), 150 mM sodium chloride. The following concentrations of preservatives were used: 3 mg/ml m-cresol, 3 mg/ml phenol.

The measured values in Table 1 are protein concentrations in micrograms/mL. As shown in Table 1, there was no loss of protein concentration in the presence of sucrose and phenol in the formulation whereas there was significant loss of protein concentration in the presence of 6 out of 9 stabilizers in the presence or absence of phenol and m-cresol.

Based on the data shown, sucrose appears to be the best stabilizer in the presence of both 2.5 mg/ml and 3.5 mg/ml of phenol in agitation, freeze-thaw and elevated temperature studies.

The various embodiments described above are provided by way of illustration only and should not be construed to limit the claimed invention. Those skilled in the art will readily recognize various modifications and changes that may be made to the claimed invention without following the example embodiments and applications illustrated and described herein, and without departing from the true spirit and scope of the claimed invention, which is set forth in the following claims.

TABLE 1

| | | | | | | | Conc | |
|---|---|---|---|---|---|---|---|---|
| | | | | | | | Change | Conc |
| | | | | | | | after 24 | Change |
| | | | | | T = 4 days | T = 24 days | days at | after 4 days |
| Formulation | [Excipient] | | Preservative | T = 0 | 25° C. | 25° C. | 25° C. | at 25° C. |
| Sucrose | 50 | mg/mL | N/A | 173 | 178 | 160 | −13 | 5 |
| Mannitol | 50 | mg/mL | N/A | 164 | 173 | 162 | −2 | 8 |
| Glycine | 50 | mg/mL | N/A | 177 | 184 | 162 | −15 | 7 |
| Arginine | 150 | mM | N/A | 170 | 166 | 174 | 4 | −5 |
| Arginine*HCl | 150 | mM | N/A | 166 | 174 | 175 | 9 | 9 |
| Sorbitol | 25 | mg/mL | N/A | 170 | 193 | 177 | 8 | 23 |
| Sodium Sulfate | 75 | mM | N/A | 166 | 169 | 166 | 0 | 3 |
| Polysorbate 20 | 0.1% | w/v | N/A | 187 | 174 | 149 | −38 | −14 |
| Sodium Chloride | 150 | mM | N/A | 186 | 175 | 171 | −15 | −12 |
| Sucrose | 50 | mg/mL | m-cresol | 171 | 108 | 74 | −97 | −63 |
| Mannitol | 50 | mg/mL | m-cresol | 163 | 96 | 71 | −92 | −67 |
| Glycine | 50 | mg/mL | m-cresol | 162 | 73 | 54 | −108 | −89 |
| Arginine | 150 | mM | m-cresol | 146 | 119 | 85 | −61 | −26 |
| Arginine*HCl | 150 | mM | m-cresol | 161 | 114 | 77 | −84 | −47 |
| Sorbitol | 25 | mg/mL | m-cresol | 156 | 82 | 60 | −96 | −74 |
| Sodium Sulfate | 75 | mM | m-cresol | 159 | 172 | 96 | −63 | 13 |
| Polysorbate 20 | 0.1% | w/v | m-cresol | 155 | 43 | 39 | −116 | −112 |
| Sodium Chloride | 150 | mM | m-cresol | 90 | 109 | 81 | −9 | 19 |
| Sucrose | 50 | mg/mL | phenol | 139 | 164 | 161 | 22 | 25 |
| Mannitol | 50 | mg/mL | phenol | 162 | 174 | 164 | 2 | 12 |
| Glycine | 50 | mg/mL | phenol | 155 | 176 | 163 | 8 | 21 |
| Arginine | 150 | mM | phenol | 171 | 153 | 142 | −29 | −17 |
| Arginine*HCl | 150 | mM | phenol | 171 | 160 | 134 | −37 | −12 |
| Sorbitol | 25 | mg/mL | phenol | 171 | 171 | 161 | −9 | 0 |
| Sodium Sulfate | 75 | mM | phenol | 174 | 150 | 104 | −70 | −25 |
| Polysorbate 20 | 0.1% | w/v | phenol | 176 | 107 | 75 | −101 | −68 |
| Sodium Chloride | 150 | mM | phenol | 176 | 131 | 107 | −69 | −45 |

What is claimed:

1. An aqueous pharmaceutical composition comprising interferon beta at a concentration of about 0.05 mg/ml to about 0.2 mg/ml, phenol at about 1 mg/ml to 6 mg/ml and sucrose at about 250 mM to 350 mM, a salt and a surfactant.

2. The composition of claim 1, wherein the interferon beta is interferon beta-1a.

3. The composition of claim 1, wherein the interferon beta is pegylated interferon beta-1a.

4. The composition of claim 1, wherein the interferon beta is interferon beta-1b.

5. The composition of claim 1, wherein the interferon beta is pegylated interferon beta-1b.

6. The composition of claim 1, wherein the interferon beta is at a concentration of 0.1 mg/ml.

7. The composition of claim 1, wherein the interferon beta is at a concentration of 0.2 mg/ml.

8. The composition of claim 1, wherein the interferon beta is at a concentration of about 0.1 to about 0.2 mg/ml.

9. The composition of claim 1, wherein the phenol is at a concentration of about 2.5 mg/ml to about 3.5 mg/ml.

10. The composition of claim 1, wherein the sucrose is at a concentration of about 250 mM to 350 mM

11. The composition of claim 1, wherein the sucrose is at a concentration of 300 mM.

12. The composition of claim 1, wherein the salt is sodium acetate.

13. The composition of claim 1, wherein the surfactant is polysorbate.

14. The composition of claim 1, wherein the pH is about 4.5 to about 6.0.

15. The composition of claim 1, wherein the pH is about 4.8.

16. The composition of claim 1, wherein the composition is stable after freeze-thaw cycling.

17. The composition of claim 1, wherein the composition is stable after agitation stress.

18. The composition of claim 1, wherein the composition is stable for at least 1 month at room temperature as measured by % monomer loss of interferon-beta.

19. The composition of claim 1, wherein the composition is stable for at least 1 month at room temperature as measured by % soluble aggregates of interferon-beta.

20. The composition of claim 1, wherein the composition is formulated as a multi-use pharmaceutical formulation.
